(19) United States
(12) Patent Application Publication
(10) Pub. No.: US 2022/0015684 A1
(43) Pub. Date: Jan. 20, 2022

(54) ELECTROPHYSIOLOGICAL SUBCORTICAL SYSTEM

(71) Applicant: Mayo Foundation for Medical Education and Research, Rochester, MN (US)

(72) Inventors: William Tatum, Ponte Vedra Beach, FL (US); Alfredo Quinones-Hinojosa, Ponte Vedra Beach, FL (US); Kaisorn L. Chaichana, Ponte Vedra Beach, FL (US)

(21) Appl. No.: 17/295,563

(22) PCT Filed: Nov. 20, 2019
(86) PCT No.: PCT/US2019/062380

§ 371 Date: May 20, 2021

Related U.S. Application Data
(60) Provisional application No. 62/770,362, filed on Nov. 21, 2018.

(51) Int. Cl. A61B 5/37; A61B 5/372; A61B 5/00; A61B 34/10; A61B 34/20; A61B 5/383; A61B 5/293

(52) U.S. Cl. CPC A61B 5/37; A61B 5/372; A61B 5/6847; A61B 34/10; A61B 2560/04; A61B 5/383; A61B 5/293; A61B 5/6882; A61B 34/20

(57) ABSTRACT

An intracranial apparatus that may be used for electrophysiological monitoring and stimulation of brain tissue of a patient. Embodiments comprise a stylet including a body section and a tip configured to separate brain tissue, a sheath including a body section having an outer surface defining a central opening configured to receive the stylet such that the tip of the stylet extends beyond the body section of the sheath, and a plurality of electrodes disposed at the outer surface of the sheath and configured to be connected to the brain tissue surrounding the sheath for stimulating and/or monitoring.

### CROSS-REFERENCE TO RELATED APPLICATION

This application is a national phase application of PCT Application No. PCT/US2019/062380, internationally filed on Nov. 20, 2019, which claims the benefit of U.S. Provisional Application No. 62/770,362, filed Nov. 21, 2018, which are herein incorporated by reference in their entireties for all purposes.

### TECHNICAL FIELD

Various aspects of the instant disclosure relate to an intracranial apparatus. In some specific examples, the disclosure concerns electrophysiological subcortical systems.

### BACKGROUND

Recent developments in neurosurgical technologies has resulted in an improved ability to perform deep-seated surgical procedures. By utilizing intracranial surgical devices capable of separating dense white matter tracks in the cephalon-caudal plane without destruction of transcortical fibers, many deep-seated lesions have become operatable for excision. This helps limit location-specific post-operative complications such as weakness, numbness, language dysfunction, or visual loss. Currently, brain MRI or other high-resolution imaging techniques are often performed prior to surgery to identify a lesion of a patient. However, the subcortical white matter tracks are deep to the surface and difficult to identify, especially during surgery. As a result, deep-seated lesions remain difficult to operate due to the depth, the surrounding eloquent regions, and the need for large dissections.

There remains a continuing need for improved intracranial surgical devices which can provide electrophysiological data to allow neurosurgeons to navigate the subcortical space with improved knowledge of the surgical areas. Technologies of these types can improve deep-seated surgical procedures operation by improving access to deeper structures and effectively make surgery safer, surgery time shortened, and post-surgery complications reduced.

### SUMMARY

Embodiments include an intracranial apparatus that may be used for electrophysiological monitoring and stimulation of brain tissue of a patient. Embodiments comprise a stylet including a body section and a tip configured to separate brain tissue; a sheath including a body section having an outer surface defining a central opening configured to receive the stylet such that the tip of the stylet extends beyond the body section of the sheath; and a plurality of electrodes disposed at the outer surface of the sheath and configured to be connected to the brain tissue surrounding the sheath (e.g., for stimulating and/or monitoring).

In examples, the plurality of electrodes may be into the sheath such that the plurality of electrodes lies substantially flush with the outer surface. The plurality of electrodes may be secured onto the outer surface of the sheath as a woven mesh wrapped around the outer surface. The plurality of electrodes may be distributed concentrically about the central opening of the sheath. Each of the plurality of electrodes may be configured to be selected as an anode or a cathode for stimulation. The plurality of electrodes may be circular and/or flat. The tip of the stylet may be substantially conical. The body section of the stylet may be substantially cylindrical. The body section of the sheath may be substantially cylindrical. The body section of the stylet may be substantially conical. The body section of the sheath may substantially conical.

Embodiments comprise a method of using an intracranial apparatus including a stylet, a sheath defining a central opening configured to receive the stylet, and a plurality of electrodes disposed at an outer surface of the sheath. Embodiments comprise inserting the apparatus into a brain such that the brain tissue of the brain is separated by the stylet and in contact with the plurality of electrodes disposed at the outer surface of the sheath; removing the stylet from the brain while the sheath remains in contact with the brain tissue; monitoring and/or stimulating the brain using the plurality of electrodes; identifying one or more sites of surgical interest based on the data from monitoring and/or stimulation of the brain; resecting brain tissue at the one or more sites of surgical interest while the sheath is in contact with the brain tissue; and removing the sheath from the brain after resecting.

In examples, identifying one or more sites of surgical interest includes differentiating non-eloquent regions from eloquent regions based on the data from monitoring the brain. Inserting the apparatus may include driving a conical tip of the stylet distally to separate brain tissue. Inserting the apparatus may include securing the sheath to a cranium of the patient. Monitoring the brain may include continuously monitoring the brain to help detect abnormality. Stimulating the brain may include selecting a first electrode as the node and a second electrode as the cathode. Stimulating the brain may be repeated at selected sites of the brain. Monitoring the brain may include monitoring at least one of motor, speech, phonetics, semantics, and visual field functions.

### BRIEF DESCRIPTION OF THE DRAWINGS

FIG. 1 shows an intracranial apparatus during a subcortical procedure, according to some examples.

FIG. 2 shows a stylet of the intracranial apparatus of FIG. 1, according to some examples.

FIG. 3A shows a top view of a sheath of the intracranial apparatus of FIG. 1, according to some examples.

FIG. 3B shows a side view of a sheath of the intracranial apparatus of FIG. 1, according to some examples.

FIG. 4 shows the intracranial apparatus of FIG. 1, according to some examples.

FIG. 5A-5D show the intracranial apparatus of FIG. 1 during a subcortical procedure, according to some examples.

FIG. 6 shows another intracranial apparatus during a subcortical procedure, according to some examples.

FIG. 7 shows a stylet of the intracranial apparatus of FIG. 6, according to some examples.

FIG. 8A shows a top view of a sheath of the intracranial apparatus of FIG. 6, according to some examples.

FIG. 8B shows a side view of a sheath of the intracranial apparatus of FIG. 6, according to some examples.

FIG. 9 shows the intracranial apparatus of FIG. 6, according to some examples.

FIG. 10 depicts an illustrative method for operating an intracranial apparatus, according to some examples.

While the disclosure is amenable to various modifications and alternative forms, specific embodiments have been shown by way of example in the drawings and are described in detail below. The disclosure, however, is not limited to the particular embodiments described. On the contrary, the disclosure is intended to cover all modifications, equivalents, and alternatives falling within the scope of the disclosure as defined by the appended claims.

### DETAILED DESCRIPTION

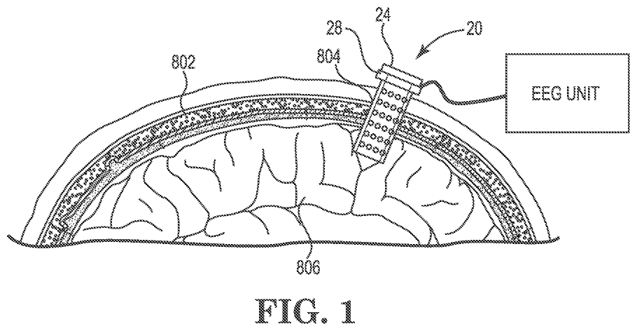

FIG. 1 shows an intracranial apparatus 20 during a subcortical procedure, according to some examples. The intracranial apparatus 20 includes a stylet 24 and a sheath 28. In various examples, the intracranial apparatus 20 is configured to be inserted into a cranium 802 of a patient via a cranial opening 804 to access the brain tissue 806 of the patient. For example, the sheath 28 of the intracranial apparatus 20 is configured to be in contact with the brain tissue 806 when the intracranial apparatus 20 is inserted. In some embodiments, the intracranial apparatus 20 is configured to be coupled to an electroencephalogram (EEG) unit, such as an EEG unit exterior to the patient. For example, the sheath 28 of the intracranial apparatus 20 is configured to be coupled to the EEG unit. In various examples, the stylet 24 is configured to be manipulated (e.g., by a surgeon) to drive the sheath 28 past the cranium 802 such that the sheath 28 is at least partially inserted past the cranial opening 804. In certain embodiments, the intracranial apparatus 20 or the sheath 28 is configured to monitor and/or stimulate the surrounding brain tissue (e.g., by a user via the EEG unit). In some embodiments, the lengths and/or diameters of the intracranial apparatus 20, the stylet 24, and/or the sheath 28 are selected at least partly based on the size of the patient.

In various embodiments, the intracranial apparatus 20 is an electrophysiological subcortical system (ESS) configured to provide electrophysiological data, such as during neurosurgeries, such as deep-seated subcortical surgical procedures. In some examples, the intracranial apparatus 20 is configured to help a user (e.g., a neurosurgeon) to navigate the subcortical space of the patient and the immediate surrounding cortical neural structures. In certain embodiments, the intracranial apparatus 20 is configured to identify abnormal neurophysiological waveforms and/or to stimulate (e.g., for functional brain mapping). In various embodiments, identifying abnormal neurophysiological activities includes analyzing (e.g., by and user) the data collected by the apparatus 20 and/or consulting a patient to which the apparatus is used on. various examples, the intracranial apparatus 20 is configured to access subcortical white matter tracts of the patient, such as tracts which are deep to the brain surface. In some embodiments, the intracranial apparatus 20 or the sheath 28 is configured to separate dense white matter tracts of the patient and to facilitate access (e.g., by providing a protected corridor for resection and/or collection) to a target site (e.g., a lesion such as a brain tumor, a vascular malformation, and/or an intracerebral hemorrhages) of the patient's brain. In certain examples, the intracranial apparatus 20 is configured to identify non-eloquent areas apart from eloquent areas prior, and/or during surgical procedures. In some embodiments, one or more sites of surgical interest are identified to be associated with the non-eloquent areas. In various embodiments, the intracranial apparatus 20 is configured to improve intra-operative localization of white matter tracts and/or refine 3-dimensional location of epileptiform activity in the subcortical space. For example, the intracranial apparatus 20 is configured to identify critical white matter tracts to avoid during surgery.

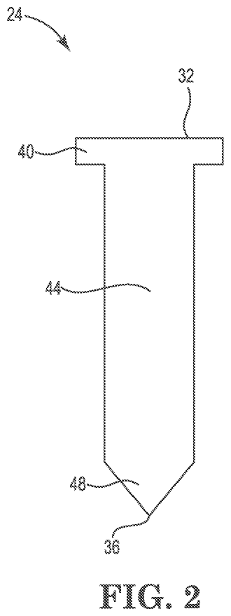

FIG. 2 shows a stylet 24 of intracranial apparatus 20, according to some examples. The stylet 24 has a proximal end 32, a distal end 36, a brim section 40 near the proximal end, a body section 44 distal to the brim section, and a tip section 48 distal to the body section. In certain examples, the brim section 40 has a diameter greater than a diameter of the body section 44 and/or greater than the cranial opening 804 (see FIG. 1). In various embodiments, the brim section 40 helps limit advancement of the stylet 24 through the cranial opening 804 (see FIG. 1) such that at least part of the stylet (e.g., the proximal end 32) is exterior to the cranium 802 (see FIG. 1). In some embodiments, the body section 44 is substantially cylindrical and extends a substantial portion of a length of the stylet 24 (e.g., more than 60%, 80%, or 90%). In various examples, the tip section 48 is substantially conical and defines an apex angle (e.g., less than 90°, 60°, or 30°) and a base diameter (e.g., substantially equal to the diameter of the body section 44). In some embodiments, the top section 48 is configured to separate the brain tissue (see FIG. 1) of the patient. In certain examples, one or more of the brim section, body section, and tip section are solid (e.g., non-hollow). In various examples, the stylet 24 includes metal such as stainless steel and/or aluminum. In some examples, the stylet 24 includes one or more slots or openings along its length.

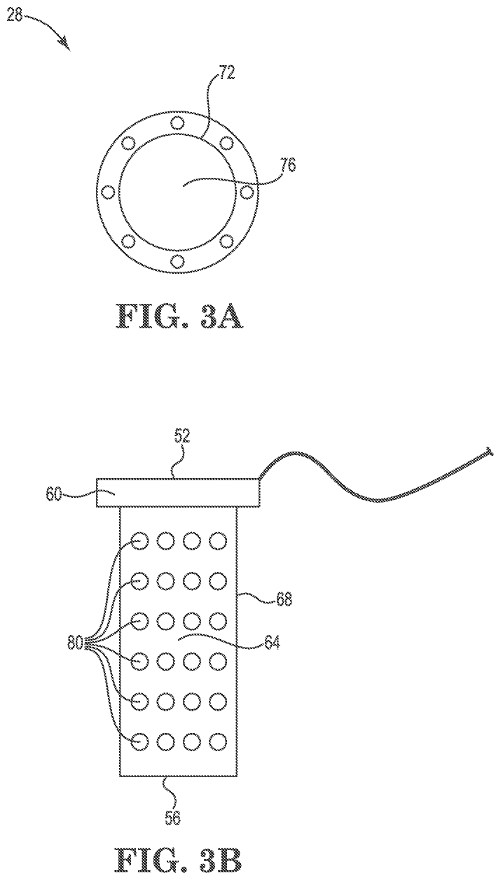

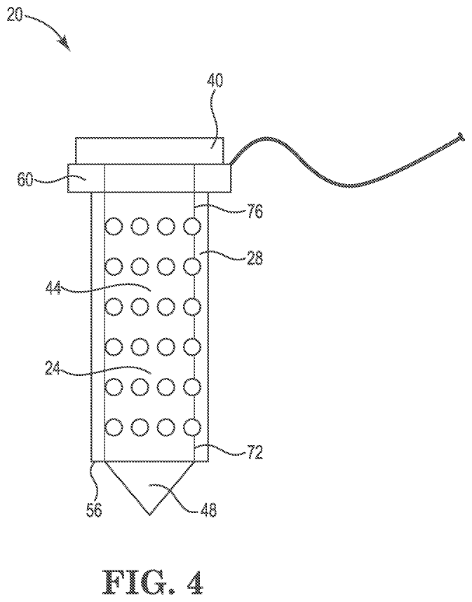

FIGS. 3A-3B show the sheath 28 of intracranial apparatus 20, according to some examples. The sheath 28 has a proximal end 52, a distal end 56, a brim section 60 near the proximal end, a body section 64 distal to the brim section 60, an outer surface 68 at the body section, an inner surface 72 extending a length of the sheath and defining a central opening 76 for receiving the stylet 24 (see FIG. 4). In some embodiments, the brim section 60 of the sheath 28 is configured to be engaged with the brim section 40 of the stylet 24 (see FIG. 2) at least during insertion of the intracranial apparatus 20 (see FIG. 1) such that advancement of the stylet leads to advancement of the sheath. In certain examples, the brim section 60 has a diameter greater than a diameter of the body section 64 and/or greater than the cranial opening 804 (see FIG. 1). In various embodiments, the brim section 60 help limit advancement of the sheath 28 through the cranial opening 804 (see FIG. 1) such that at least part of the sheath (e.g., the proximal end 52) is exterior to the cranium 802 (see FIG. 1). For example, the brim section 60 is configured to engage the cranium 802 (see FIG. 1) near the cranial opening 804 (see FIG. 1) at the deepest level of advancement. In some embodiments, the body section 64 is substantially tubular and extends a substantial portion of a length of the sheath 28 (e.g., more than 60%, 80%, or 90%). In some examples, the sheath 28 is 50 mm to 100 mm in length (e.g., 50 mm, 60 mm, or 75 mm) and has a diameter of 10 mm to 30 mm (e.g., 11 mm, 13.5 mm, or 20 mm) at the body section 64. In certain examples, the sheath 28 includes plastic such as clear plastic and/or is configured to permit access to a target site in the brain (via the central opening 76). In some embodiments, the sheath 28 includes one or more openings or slots configured for a surgical tool to be extended through from the central opening 76 to the brain tissue exterior of the sheath.

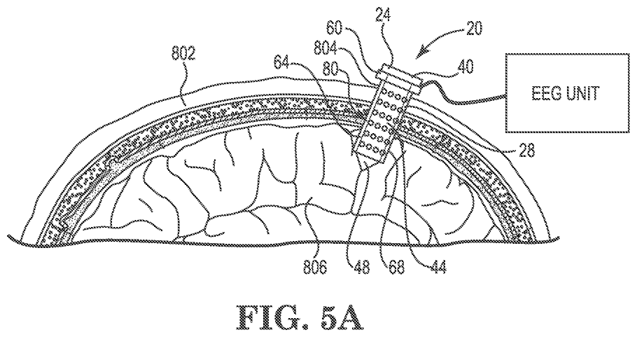

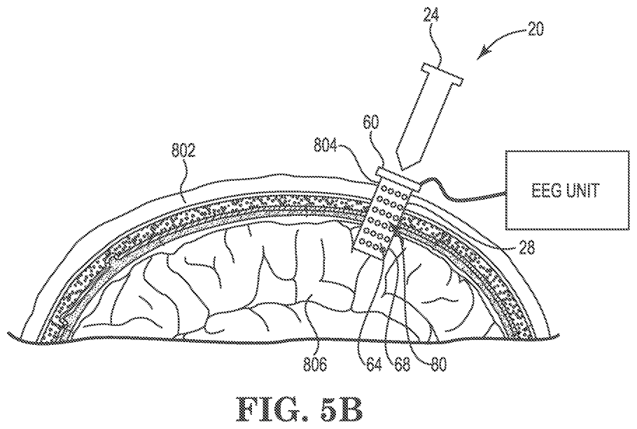

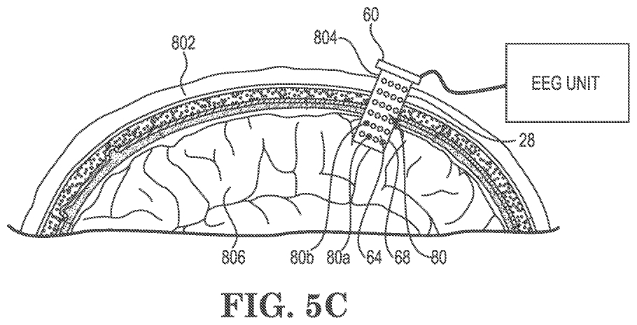

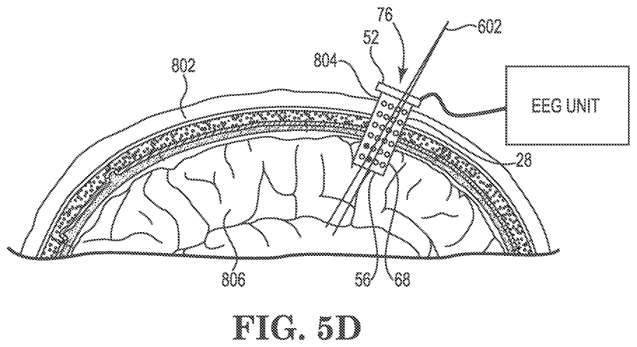

In various embodiments, the sheath 28 further includes a plurality of electrodes 80 (e.g., an array of electrodes) disposed at the body section 64. For example, the plurality of electrodes 80 are positioned near or at the outer surface 68 and configured to contact brain tissue 804 surrounding the sheath when inserted (see FIG. 5C). In certain examples, the plurality of electrodes 80 is configured to stimulate and/or monitor brain tissues surrounding the sheath 28 (e.g., via the EEG unit). In some examples, the plurality of electrodes 80 is substantially uniformly distributed having a substantially constant electrode-to-electrode spacing (e.g., concentrically about the central opening 76). In other examples, the plurality of electrodes 80 is non-uniformly distributed such that resolution for stimulation and/or monitoring is higher at certain regions of the sheath 28. In various embodiments, the plurality of electrodes 80 includes 32 to 256 electrodes with an electrode-to-electrode spacing between 2.5 mm to 10 mm (e.g., a 5 mm spacing). For example, the plurality of electrodes 80 includes 8 rows of electrodes with each row configured to be positioned at different depths when the sheath 28 is inserted in the patient's brain. Such arrangement of electrodes at various depths can enable monitoring and/or stimulation at various depths. In certain examples, the plurality of electrodes 80 are substantially circular and flat and sized between 2 mm to 5 mm. In various embodiments, the plurality of electrodes 80 is secured onto the outer surface 68 of the sheath 28, such as being woven into a mesh and wrapped around the outer surface. In some embodiments, the plurality of electrodes 80 is embedded into the sheath 28 such that the plurality of electrodes lies substantially flush with the outer surface 68. For example, the plurality of electrodes 80 is imbedded in a protective material (e.g., Silastic®). In certain examples, the plurality of electrodes 80 includes stainless steel, platinum, graphene, and/or a conductive polymer.

In some examples, the plurality of electrodes 80 are configured to stimulate and monitor (e.g., record) brain tissue by being electrically connected to the EEG unit (see FIG. 5C) via wired (e.g., via a jack box) and/or wireless connections. For example, the plurality of electrodes 80 includes electrodes independently connected to the EEG unit (see FIG. 5C). In certain embodiments, the plurality of electrodes 80 includes electrodes configured to monitor brain tissue simultaneously. In certain examples, each electrode of the plurality of electrodes 80 includes a marking element (e.g., a number, a color code) to help orient the sheath and identify spatial positions of the plurality of electrodes and/or the target sites. In various embodiments, the marking element are used to indicate which electrodes are where target site is to help improve surgical precision. In certain examples, the sheath 28 includes lighting elements configured to provide illumination to aid with the surgical procedure.

FIG. 4 shows the intracranial apparatus 20 configured to be inserted into the patient's brain via the cranial opening 804 of the cranium 802 such that brain tissue 806 can be accessed (see FIG. 1), according to some examples. As shown, the stylet 24 is configured to be coupled to the sheath 28 such that the brim section 40 of the stylet is coupled (e.g., in contact) to the brim section 60 of the sheath, the body section 44 of the stylet is received by the central opening 76 of the sheath, and the tip section 48 of the stylet is extended beyond the distal end 56 of the sheath. In certain examples, the inner surface 72 of the sheath 28 is configured to contact the body section 44 of the stylet 24 during insertion of the intracranial apparatus 20. In some embodiments, the sheath 28 is shorter or equal to the length of the body section 44 of the stylet 24.

FIG. 5A-5D show the intracranial apparatus 20 during a subcortical procedure, according to some examples. As shown in FIG. 5A, the stylet 24 and the sheath 28 are coupled during insertion of the intracranial apparatus 20 such that manipulation of the stylet (e.g., by a user) can lead to advancement of the sheath. The intracranial apparatus 20 configured to be inserted into the cranium 802 via the cranial opening 804. In various examples, the tip section 48 of the stylet 24 is driven distally into the patient's brain to help separate the brain tissue 806. In some embodiments, the body section 64 of the sheath 28, which is supported by the body section 44 of the stylet 24, is configured to be positioned between the separated brain tissue 806 such that the plurality of electrodes 80 at the outer surface 68 of the sheath 28 is coupled (e.g., electrically) to the brain tissue for stimulation and/or monitoring. In certain examples, the brim section 40 of the stylet 24 and the brim section 60 of the sheath 28 are configured (e.g., sized) to be prevented from passing through the cranial opening 804 such that at least part of the intracranial apparatus 20 remains exterior to the cranium 802 when inserted. In some embodiments, the brim section 60 of the sheath 28 is configured to be secured (e.g., via fasteners) to the cranium 802 to aid stability.

As shown in FIG. 5B, once the apparatus 20 (e.g., the sheath 28) is inserted, the stylet 24 of the intracranial apparatus is configured to be decoupled from the sheath 28 such that the central opening 76 of the sheath is exposed or freed for entry. In various examples, the sheath 28 remains secured to the patient's brain and/or cranium 802 during removal of the stylet 24 such that the plurality of electrodes 80 at the outer surface 68 of the body section 64 remain coupled (e.g., electrically) to the brain tissue 806 for stimulation and/or monitoring.

As shown in FIG. 5C, once the plurality of electrodes 80 is coupled to the brain tissue 806, the sheath 28 is configured to stimulate and/or monitor the brain tissue. In some examples, a pair of electrodes from the plurality of electrodes 80 are selected as bi-polar pair and are connected to a stimulator to stimulate the brain tissue. For example, the plurality of electrodes 80 includes a first electrode 80a selected as the anode and a second electrode 80b selected as the cathode. In various examples, each electrode of the plurality of electrodes 80 can be selected as an anode or a cathode of a bi-polar pair. In some embodiments, the electrodes of the plurality of electrodes 80 which are not used for stimulation are configured to monitor brain tissue (e.g., continuously throughout the surgery). In certain examples, all of the plurality of electrodes 80 are configured to first monitor the brain tissue to identify abnormal neurophysiological waveforms at sites of surgical interest then stimulate by selecting bi-polar electrode pairs near the sites of surgical interest. In some embodiments, one or more of the sites of surgical interest are identified to be resected (e.g., by a surgeon).

As shown in FIG. 5D, once the stylet is removed from the sheath 28, the central opening 76 is configured to provide a corridor or path of access for a surgical tool 602 to enter the cranium 802. For example, the sheath 28 is configured such that the surgical tool 602 can access the brain tissue 806 (e.g., for resection and/or collection) by entering the sheath from the proximal end 52 and extends beyond the distal end 56. In some embodiments, the sheath 28 is configured such that the surgical tool 602 can extend between the outer surface 68 and the surrounding brain tissue 806 for resection and/or collection. In various examples, the sheath 28 is configured to stimulate and/or monitor (e.g., simultaneously) the brain tissue 806 while the surgical tool 602 is manipulated for dissection and/or collection of brain tissue. In certain embodiments, the surgical tool 602 includes a navigation device and/or an imaging device.

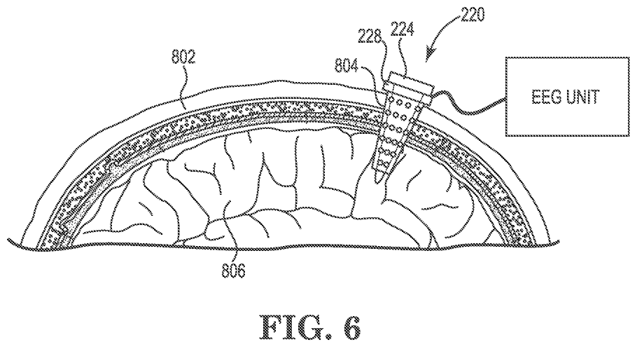

FIGS. 6-9 show another intracranial apparatus 220, according to some examples. The intracranial apparatus 220 may be substantially similar to the intracranial apparatus 20 and may include features, functions, and/or elements of intracranial apparatus 20. As shown in FIG. 6, the intracranial apparatus 220 includes a stylet 224 and a sheath 228. In various examples, the intracranial apparatus 220 is configured to be inserted into a cranium 802 of a patient via a cranial opening 804 to access the brain tissue 806 of the patient. In some embodiments, the conical intracranial apparatus 220 is configured for variable-depth access to subcortical organ resection, stimulation, and/or monitoring.

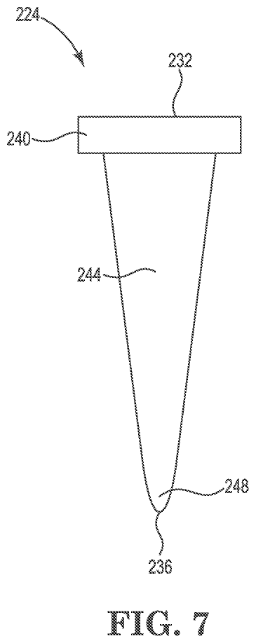

As shown in FIG. 7, the stylet 224 has a proximal end 232, a distal end 236, a brim section 240 near the proximal end, a body section 244 distal to the brim section, and a tip 248 distal to the body section. In certain examples, the body section 244 is substantially conical and extends a substantial portion of a length of the stylet 224 (e.g., more than 60%, 80%, or 90%). For example, the body section 244 extends from the brim section 240 to the tip 248 and defines an apex angle (e.g., less than 90°, 60°, or 30°) and a base diameter (e.g., smaller than the brim section and cranial opening). In some embodiments, the tip 248 and the body section 244 are configured to separate the brain tissue (see FIG. 6) of the patient.

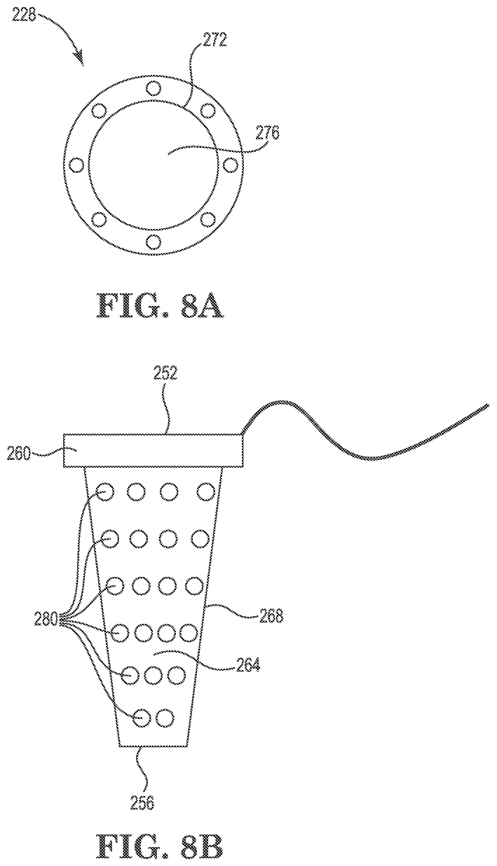

FIGS. 8A-8B show the sheath 228 has a proximal end 252, a distal end 256, a brim section 260 near the proximal end, a body section 264 distal to the brim section 260, an outer surface 268 at the body section, an inner surface 272 extending a length of the sheath and defining a central opening 276 for receiving the stylet 224 (see FIG. 7). In some embodiments, the body section 264 is substantially conical (e.g., a frustum) and extends a substantial portion of a length of the sheath 228 (e.g., more than 60%, 80%, or 90%). For example, the body section 264 has a first diameter near the proximal end 252 or the brim section 260 and a second diameter smaller than the first diameter near the distal end 256. In various embodiments, the sheath 228 further includes a plurality of electrodes 280 (e.g., an array of electrodes) disposed at the body section 264 for stimulation and/or monitoring of brain tissue.

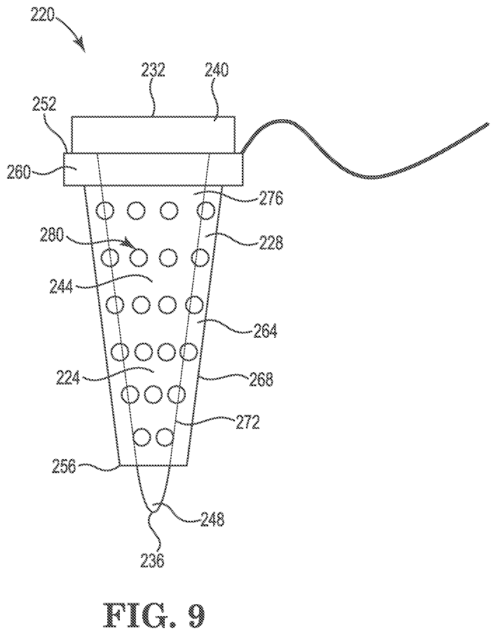

FIG. 9 shows the intracranial apparatus 220 configured to be inserted into the patient's brain via the cranial opening 804 of the cranium 802 such that brain tissue 806 can be accessed (see FIG. 6), according to some examples. As shown, the stylet 224 is configured to be coupled to the sheath 228 such that the brim section 240 of the stylet is coupled (e.g., in contact) to the brim section 260 of the sheath, the body section 244 of the stylet is received by the central opening 276 of the sheath, and the tip 248 of the stylet is extended beyond the distal end 256 of the sheath. In certain examples, the inner surface 272 of the sheath 228 is configured to contact the body section 244 of the stylet 224 during insertion of the intracranial apparatus 220. In some embodiments, the sheath 228 is shorter or equal to the length of the body section 244 of the stylet 224.

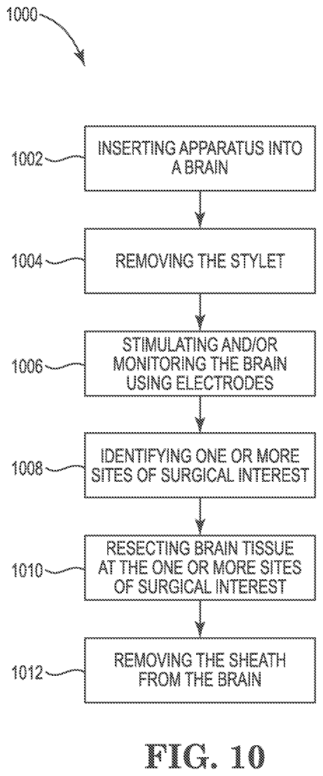

FIG. 10 depicts an illustrative method 1000 for operating an intracranial apparatus (e.g., intracranial apparatus 20 and/or intracranial apparatus 220), according to some examples. The method 1000 includes inserting 1002 the apparatus into a brain such that the brain tissue of the brain is separated by a stylet (e.g., stylet 24 or stylet 224) and in contact with a plurality of electrodes disposed at an outer surface of a sheath (e.g., sheath 28 or sheath 228), removing 1004 the stylet from the brain while the sheath remains in contact with the brain tissue, monitoring and/or stimulating 1006 the brain using the plurality of electrodes, identifying 1008 one or more sites of surgical interest based on the data from monitoring and/or stimulation of the brain, resecting 1010 brain tissue at the one or more sites of surgical interest while the sheath is in contact with the brain tissue, and removing 1012 the sheath from the brain after resecting. In various embodiments, the method 1000 is performed during a surgical procedure and may further include inducing and intubating the patient, performing craniotomy, and surgical closure of the dura, bone, and skin.

In some examples, inserting 1002 the intracranial apparatus into a brain includes coupling a stylet with a sheath and advancing the stylet into a cranial opening (e.g., cranial opening 804) of a cranium (e.g., cranium 802) of the patient such that the sheath is advanced into the brain and be in contact with the brain tissue (e.g., brain tissue 806) of the patient. In various embodiments, advancing the stylet includes driving a tip (e.g., tip section 48 or tip 248) to separate white matter of the brain and guiding a body section (e.g., body section 64 or body section 264) into the separation (e.g., to prevent the brain from herniating into the center of the sheath). In certain examples, the stylet is advanced at most to when a brim section (e.g., brim section 60 or brim section 260) of the sheath comes into contact with the cranium. In some embodiments, inserting 1002 the intracranial apparatus includes securing the sheath to the cranium (e.g., via fasteners). In various examples, the intracranial apparatus is inserted such that a plurality of electrodes (e.g., electrodes 80 or electrodes 280) are coupled to the brain tissue. In certain embodiments, removing 1004 the stylet from the brain includes removing the stylet from the sheath such that a central opening (e.g., central opening 76 or central opening 276) of the sheath is exposed (e.g., to a user).

In some examples, monitoring 1006 the brain using electrodes includes electrophysiological monitoring of the cortical and subcortical space. In some embodiments, each electrode of the plurality of electrodes is independently coupled to an electroencephalogram (EEG) unit and configured to be used for constant monitoring of the brain. In certain embodiments, monitoring 1006 the brain using electrodes includes performing baseline measurements and recording for identifying abnormalities (e.g., abnormal waveforms). In various examples, monitoring 1006 the brain using electrodes includes evaluating responses to electrical stimulation at or near sites of surgical interest. In some embodiments, evaluating responses includes determining whether the stimulation is positive or negative and/or providing information (e.g., to a user) regarding the spatial position of a site of surgery interest. For example, if a position stimulation is determined at a certain depth, the region superficial to the depth identified near the sheath is to be removed. In some embodiments, monitoring 1006 the brain is performed before, during and/or after the stimulating 1006 and/or the resecting 1010 the brain. In various embodiments, monitoring 1006 the brain is performed continuously (e.g., during stimulation and/or resection of brain tissue)

In some examples, stimulating 1006 the brain using electrodes includes using a neurostimulator. In various embodiments, stimulating 1006 the brain includes selecting a bi-polar electrode pair (e.g., an anode and a cathode) for stimulation. In certain examples, the electrode pair are selected near or at the site of surgical interest (e.g., based on the monitoring result). For example, stimulating 1006 the brain using electrodes includes attaching a pair of electrodes to the neurostimulator. In various embodiments, various stimulations may be carried out simultaneously using a plurality of pairs of electrodes. In some examples, the stimulation step and the monitoring step are performed simultaneously. In certain embodiments, repeated stimulation at selected sites is performed. In various examples, the stimulation uses a charge density less than 30 μC/mm³. In some embodiments, stimulation helps with functional brain mapping. In some examples, identifying 1008 one or more sites of surgical interest includes differentiating non-eloquent regions from eloquent regions based on the data from monitoring the brain. In various embodiments, the one or more sites of surgical interest includes identifying one or more sites for stimulation 1006. In certain examples, the sites for stimulation and/or resection are identified prior, during, or after each stimulation and/or resection.

In some examples, resecting (or removing) 1010 brain tissue includes inserting a surgical tool (e.g., surgical tool 602) through the sheath, such as from the proximal end (e.g., proximal end 52 or proximal end 252) via the central opening and through the distal end (e.g., distal end 56 or distal end 256) and/or through the slots/holes on the side. In certain embodiments, resecting 1008 brain tissue includes inserting the surgical tool around the sheath and between the sheath and the brain tissue surrounding the sheath. In various examples, resecting 1008 brain tissue includes inserting a navigation device and/or an imaging device. In some embodiments, resecting 1008 brain tissue is performed simultaneously and/or intermittently with the monitoring 1004 and stimulating 1006 steps. In some examples, resecting 1008 brain tissue includes resecting non-eloquent tissue or pathological tissue (e.g., identified by the monitoring 1006 step).

In various embodiments, the stylet and sheath are advanced into the brain to access one or more regions of the brain identified to be resected (e.g., a tumor). In some examples, monitoring and stimulation of the brain can be performed with the plurality of electrodes when the patient is awake or asleep. In certain embodiments, information related to motor, sensory, or language functions are monitored. In some embodiments, the sheath acts as a depth gauge and helps identify the brain region to be resected.

In certain examples, a surgical procedure using an intracranial apparatus (e.g., intracranial apparatus 20 and/or intracranial apparatus 220) includes inducing and intubating the patient and positing the patient per routine (e.g., with Mayfield skull clamps); attaching and registering one or more navigation devices to the Mayfield frame; plan incision based on entry into the lesion; draping the patient; making an incision and performing a craniotomy; opening the dura; operating the navigation devices for cortical and/or subcortical monitoring (e.g., via the cortex or the sulcus); determining the depth of surgical sites; removing the stylet (e.g., while the sheath prevents the brain from herniating into the center of the sheath); performing electrophysiological monitoring of the cortical and/or subcortical space (e.g., monitor motor, speech, phonetics, semantics, and/or visual field functions); stimulating cortical and/or subcortical letrozole; identifying areas causing deficits (e.g., predicated on the tumor location); withdrawing the apparatus after resection is completed; and/or closing the dura, bone, and skin in standard fashion.

In certain embodiments, an electrophysiological procedure using an intracranial apparatus (e.g., intracranial apparatus 20 and/or intracranial apparatus 220) includes placing the apparatus at regions of the brain that is of surgical interest; removing the stylet after the sheath (e.g., an electrophysiological conical grid system) is advanced to the desired position (e.g., approximate to the depth of a targeted surgical site or lesion); ensuring (e.g., connecting) the connection between the sheath and an EEG machine via a jack box; performing a baseline (e.g., dense array) EEG from the plurality of electrodes (e.g., on the sheath); displaying the baseline EEG on the EEG machine monitor; recording to help identify the presence of epileptiform abnormalities (e.g., for 5 minute, continuously or intermittently); electrically stimulating at or near the sites of surgical interest using the selected electrodes of the plurality of electrodes via a neurostimulator; evaluating endpoints during serial stimulations for clinical signs or symptoms (e.g., speech arrest, motor weakness or jerking, sensory paresthesia, visual phosphenes or scotomata), after discharge or EEG seizures, and maximal safe current (e.g., with charge density below 30 μC/mm³); removing the sheath, performing surgical closure; and/or transferring the patient (e.g., to a recovery room).

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

1. An intracranial apparatus for providing electrophysiological monitoring and stimulation of brain tissue of a patient, the apparatus comprising:
a stylet including a body section and a tip configured to separate brain tissue;
a sheath including a body section having an outer surface and defines a central opening configured to receive the stylet such that the tip of the stylet extends beyond the body section of the sheath; and
a plurality of electrodes disposed at the outer surface of the sheath and configured to be connected to the brain tissue surrounding the sheath for stimulating and monitoring.

2. The apparatus of claim 1, wherein the plurality of electrodes is into the sheath such that the plurality of electrodes lies substantially flush with the outer surface.

3. The apparatus of claim 1, wherein the plurality of electrodes is secured onto the outer surface of the sheath as a woven mesh wrapped around the outer surface.

4. The apparatus of claim 1, wherein the plurality of electrodes is distributed concentrically about the central opening of the sheath.

5. The apparatus of claim 1, wherein each of the plurality of electrodes is configured to be selected as an anode or a cathode for stimulation.

6. The apparatus of claim 1, wherein the plurality of electrodes is circular and flat.

7. The apparatus of claim 1, wherein the tip of the stylet is substantially conical.

8. The apparatus of claim 1, wherein the body section of the stylet is substantially cylindrical.

9. The apparatus of claim 8, wherein the body section of the sheath is substantially cylindrical.

10. The apparatus of claim 1, wherein the body section of the stylet is substantially conical.

11. The apparatus of claim 10, wherein the body section of the sheath is substantially conical.

12. A method of using an intracranial apparatus including a stylet, a sheath defining a central opening configured to receive the stylet, and a plurality of electrodes disposed at an outer surface of the sheath, the method comprising:
inserting the apparatus into a brain such that the brain tissue of the brain is separated by the stylet and in contact with the plurality of electrodes disposed at the outer surface of the sheath;
removing the stylet from the brain while the sheath remains in contact with the brain tissue;
monitoring and/or stimulating the brain using the plurality of electrodes;
identifying one or more sites of surgical interest based on the data from monitoring and/or stimulation of the brain;
resecting brain tissue at the one or more sites of surgical interest while the sheath is in contact with the brain tissue; and
removing the sheath from the brain after resecting.

13. The method of claim 12, wherein identifying one or more sites of surgical interest includes differentiating non-eloquent regions from eloquent regions based on the data from monitoring the brain.

14. The method of claim 12, wherein inserting the apparatus includes driving a conical tip of the stylet distally to separate brain tissue.

15. The method of claim 12, wherein inserting the apparatus includes securing the sheath to a cranium of the patient.

16. The method of claim 12, wherein monitoring the brain includes continuously monitoring the brain to help detect abnormality.

17. The method of claim 12, wherein stimulating the brain includes selecting a first electrode as the node and a second electrode as the cathode.

18. The method of claim 12, wherein stimulating the brain is repeated at selected sites of the brain.

19. The method of claim 12, wherein monitoring the brain includes monitoring at least one of motor, speech, phonetics, semantics, and visual field functions.
